(19) United States
(12) Patent Application Publication
(10) Pub. No.: US 2024/0245336 A1
(43) Pub. Date: Jul. 25, 2024

(54) BIDIRECTIONAL STRETCHABLE NERVE FIBER INTERFACE AND MANUFACTURING METHOD THEREOF

(71) Applicant: RESEARCH & BUSINESS FOUNDATION SUNGKYUNKWAN UNIVERSITY, Suwon-si (KR)

(72) Inventors: Donghee SON, Suwon-si (KR); Soojung AN, Suwon-si (KR); Yewon KIM, Suwon-si (KR)

(73) Assignee: RESEARCH & BUSINESS FOUNDATION SUNGKYUNKWAN UNIVERSITY, Suwon-si (KR)

(21) Appl. No.: 18/417,165

(22) Filed: Jan. 19, 2024

(30) Foreign Application Priority Data
Jan. 20, 2023 (KR) 10-2023-0008713

(51) Int. Cl. A61B 5/268; A61B 5/27; A61B 5/294

(52) U.S. Cl. CPC A61B 5/268; A61B 5/27; A61B 5/294; A61B 2562/0209; A61N 1/0551

(57) ABSTRACT

An embodiment of the disclosure provides a bidirectional stretchable nerve interface and manufacturing method thereof. According to an embodiment of the disclosure, a bidirectional stretchable nerve interface may provide a fiber-based bidirectional stretchable nerve interface that is soft, has high electrical and mechanical durability, and has excellent stimulation and neural signal measurement performance.

### CROSS REFERENCE TO RELATED APPLICATION

The present application claims priority to Korean Patent Application No. 10-2023-0008713, filed Jan. 20, 2023, the entire contents of which is incorporated herein for all purposes by this reference.

### BACKGROUND OF THE INVENTION

### Field of the Invention

The disclosure relates to a bidirectional stretchable nerve fiber interface with excellent electrical and mechanical properties and a method of manufacturing the same.

### Description of the Related Art

Conventional metal electrodes are rigid and have modulus mismatching with those of tissues, causing inflammatory reactions and fibrosis, making it difficult to use them as materials for implantable devices.

To solve the above problem, much research is being conducted on implantable devices using materials with properties similar to those of tissues, but these materials have difficulty maintaining their shape when used for long periods of time or have limitations in maintaining electrical or mechanical properties.

In addition, when all the previously mentioned materials are used for such a long period of time, there are problems with unstable mechanical and electrical performance.

Therefore, many challenges still remain to achieve stretchable nerve interfaces.

### Documents of Related Art

(Patent Document 1) KR Registered Patent No. 10S-1900472

### SUMMARY OF THE INVENTION

The technical object to be achieved by the disclosure is to provide a stretchable nerve interface with increased biocompatibility and electrical and mechanical durability using stretchable fabric-based substrate with inert metal-based fiber nerve electrode, and a method for manufacturing a stretchable nerve interface that does not require a separate adhesive material when applied to the nerve using self-bonding property.

The technical objects to be achieved by the disclosure are not limited to the technical objects mentioned above, and other technical objects not mentioned may be clearly understood by those skilled in the art from the following descriptions.

In order to achieve the above object, an embodiment of the disclosure provides a bidirectional stretchable nerve interface.

The bidirectional stretchable nerve interface according to an embodiment of the disclosure comprises a stretchable fabric-based substrate in which a stretchable fabric is located between respective layers of a plurality of self-healing polymer films; and a fiber-based nerve electrode which is located on the fabric-based substrate and is made of a fiber coated with a conductive composite ink containing a self-healing polymer and metal-based fillers.

In addition, according to an embodiment of the disclosure, the fabric-based substrate and the fiber-based nerve electrode may be self-bonded.

In addition, according to an embodiment of the disclosure, the stretchable fabric may include one or more selected from the group consisting of polypropylene, nylon, polyvinylidene fluoride (PVDF), viscose, ultra-high-molecular-weight polyethylene (UHMWPE), polytetrafluoroethylene (PTFE), polyglycolic acid (PGA), catgut, catgut chromic, polyglactin 910, silk, poliglecaprone, polydioxanone, polyurethane, spandex, polyglycolic acid, and polyester.

In addition, according to an embodiment of the disclosure, the self-healing polymer may be an elastomer material whose backbone is any one selected from the group consisting of polydimethylsiloxane (PDMS), polyethyleneoxide (PEO), perfluoropolyether (PFPE), polybutylene (PB), poly(ethylene-co-1-butylene), poly(butadiene), hydrogenated poly(butadiene), poly(ethylene oxide)-poly(propylene oxide) block copolymer, random copolymer, and poly(hydroxyalkanoate).

In addition, according to an embodiment of the disclosure, the fiber may be a composite that includes one or more fibers selected from the group consisting of polypropylene, nylon (polyamide), PVDF, viscose, UHMWPE, PTFE, PGA, catgut, catgut chromic, polyglactin 910, silk, poliglecaprone, polydioxanone, polyurethane, spandex, polyglycolic acid and polyester.

In addition, according to an embodiment of the disclosure, the metal-based fillers may be a composite in which a metal nanoshell is coated on a metal flake.

In addition, according to an embodiment of the disclosure, the metal flake may include one or more metals selected from the group consisting of silver, copper, and aluminum.

In addition, according to an embodiment of the disclosure, the metal nanoshell may include one or more metals selected from the group consisting of platinum, gold, and iridium.

In addition, according to an embodiment of the disclosure, a thickness of the metal nanoshell may be 1 nm to 50 nm.

In addition, according to an embodiment of the disclosure, a strain rate of the bidirectional stretchable nerve interface may be 10% or more.

In order to achieve the above object, another embodiment of the disclosure provides a method for manufacturing a bidirectional stretchable nerve interface.

The method for manufacturing a bidirectional stretchable nerve interface according to an embodiment of the disclosure may comprise the steps of preparing a stretchable fabric-based substrate by coating a self-healing polymer film to surround a stretchable fabric; preparing conductive composite ink by mixing metal fillers and self-healing polymer solution; Preparing coated nerve electrode by dipping a fiber into the conductive composite ink to surround a fiber; and preparing a stretchable nerve interface by combining the fabric-based substrate and the fiber-based nerve electrode.

In addition, according to an embodiment of the disclosure, in the step of preparing the stretchable fabric-based substrate, the stretchable fabric may include one or more selected from the group consisting of polypropylene, nylon, polyvinylidene fluoride (PVDF), viscose, ultra-high-molecular-weight polyethylene (UHMWPE), polytetrafluoroethylene (PTFE), polyglycolic acid (PGA), catgut, catgut chromic, polyglactin 910, silk, poliglecaprone, polydioxanone, polyurethane, spandex, polyglycolic acid, and polyester.

In addition, according to an embodiment of the disclosure, in the step of preparing the stretchable substrate, the self-healing polymer may be an elastomer material whose backbone is any one selected from the group consisting of polydimethylsiloxane (PDMS), polyethyleneoxide (PEO), perfluoropolyether (PFPE), polybutylene (PB), poly(ethylene-co-1-butylene), poly(butadiene), hydrogenated poly(butadiene), poly(ethylene oxide)-poly(propylene oxide) block copolymer, random copolymer, and poly(hydroxyalkanoate).

In addition, according to an embodiment of the disclosure, in the step of preparing the conductive composite ink, the metal-based fillers may be a composite in which a metal nanoshell is coated on a metal flake.

In addition, according to an embodiment of the disclosure, the metal flake may include one or more metals selected from the group consisting of silver, copper, and aluminum.

In addition, according to an embodiment of the disclosure, the metal nanoshell may include one or more metals selected from the group consisting of platinum, gold, and iridium.

In addition, according to an embodiment of the disclosure, a thickness of the metal nanoshell may be 1 nm to 50 nm.

In addition, according to an embodiment of the disclosure, in the step of preparing the fiber-based nerve electrode, the fiber may be a composite that includes one or more fibers selected from the group consisting of polypropylene, nylon (polyamide), PVDF, viscose, UHMWPE, PTFE, PGA, catgut, catgut chromic, polyglactin 910, silk, poliglecaprone, polydioxanone, polyurethane, spandex, polyglycolic acid and polyester.

In addition, according to an embodiment of the disclosure, in the step of preparing the stretchable nerve interface, the fabric-based substrate and the fiber-based nerve electrode may be self-bonded.

### BRIEF DESCRIPTION OF THE DRAWINGS

FIG. 1 is a schematic diagram showing a bidirectional stretchable nerve interface according to an embodiment of the disclosure.

FIG. 2 shows (a) a front view and cross-sectional view of a fiber-based nerve electrode, and (b) a cross-sectional view of a bidirectional stretchable nerve interface according to an embodiment of the disclosure.

FIG. 3 is a flowchart showing a method for manufacturing a bidirectional stretchable nerve interface according to an embodiment of the disclosure.

FIG. 4 is an actual photograph of a bidirectional stretchable nerve interface according to an embodiment of the disclosure.

FIG. 5 is a graph showing a change in resistance of a nerve electrode according to an embodiment of the disclosure.

FIG. 6 is a photograph showing before (A) and after (B) applying electrical stimulation to rat with a fabric-based stretchable nerve interface according to an embodiment of the disclosure to a rat.

FIG. 7 is a graph showing a result of neural signal measurement according to various mechanical stimulation intensities according to an embodiment of the disclosure.

### DETAILED DESCRIPTION OF THE INVENTION

Hereinafter, the disclosure will be explained with reference to the accompanying drawings. The disclosure, however, may be modified in various different ways, and should not be construed as limited to the embodiments set forth herein. Also, in order to clearly explain the disclosure, portions that are not related to the disclosure are omitted, and like reference numerals are used to refer to like elements throughout.

Throughout the specification, when an element is referred to as being “connected (accessed, contacted, coupled) to” another element, this includes “direct connection” and “indirect connection” via an intervening element. Also, when a certain part “includes” a certain component, other components are not excluded unless explicitly described otherwise, and other components may in fact be included.

The terms used in the following description are intended to merely describe specific embodiments, but not intended to limit the disclosure. An expression of the singular number includes an expression of the plural number, so long as it is clearly read differently. The terms such as “include” and “have” are intended to indicate that features, numbers, steps, operations, elements, components, or combinations thereof used in the following description exist and it should thus be understood that the possibility of existence or addition of one or more other different features, numbers, steps, operations, elements, components, or combinations thereof is not excluded.

Hereinafter, embodiments of the disclosure will be described in detail with reference to the accompanying drawings.

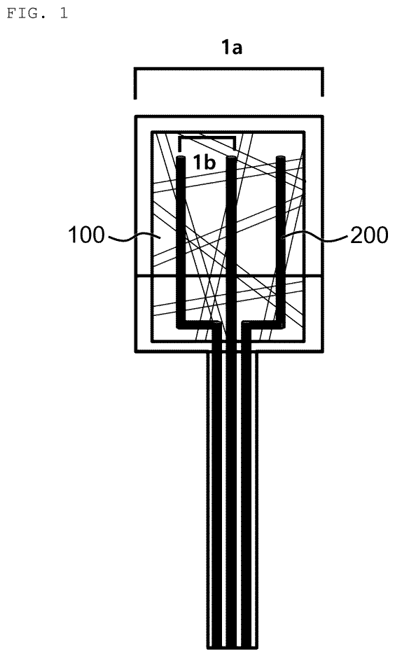

Here, a bidirectional stretchable nerve interface according to an embodiment of the disclosure will be described with reference to FIGS. 1, 2, and 4.

Conventional metal electrodes are rigid and have modulus mismatching with those of tissues, causing inflammatory reactions and fibrosis, making it difficult to use them as materials for implantable devices. Much research has been conducted on implantable devices using materials with mechanical properties similar to those of tissues, but these materials have limitations in maintaining electrical or mechanical properties when used for long periods of time. When these materials are used for a long time, there are problems with limited elasticity, deterioration of mechanical and electrical performance due to shape deformation and damage, but the structure in which the fiber is coated with conductive composite ink according to the disclosure can overcome these problems. Concretely, in order to solve the above problem, a nerve interface of the disclosure is characterized by matching modulus with the tissues by combining a polymer with similar properties to the skin and fiber-based electrodes that can self-healing, and without ion release and oxidation by including a metal composite such as silver-platinum, thereby improving biocompatibility and increasing charge delivery capacity.

FIG. 1 is a schematic diagram showing a bidirectional stretchable nerve interface according to an embodiment of the disclosure.

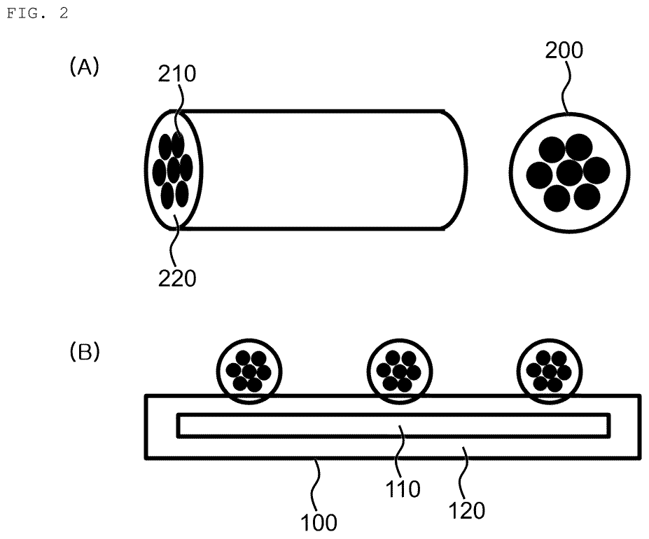

FIG. 2 shows (a) a front view and cross-sectional view of a fiber-based nerve electrode, and (b) a cross-sectional view of a bidirectional stretchable nerve interface according to an embodiment of the disclosure.

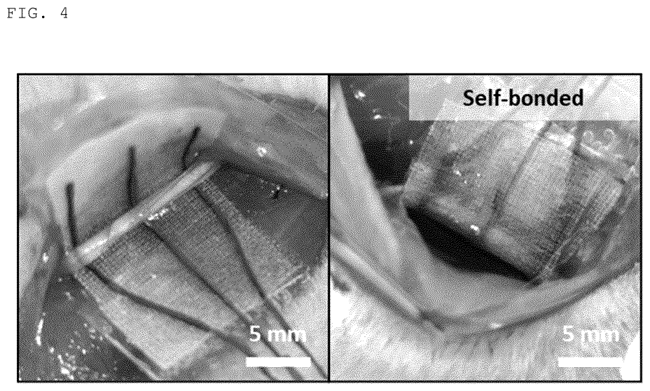

FIG. 4 is an actual photograph of a bidirectional stretchable nerve interface according to an embodiment of the disclosure, which is wrapped around a nerve.

A bidirectional stretchable nerve interface according to an embodiment of the disclosure comprises a fabric-based substrate 100 in which a stretchable fabric is located between respective layers of a plurality of self-healing polymer films; and a fiber-based nerve electrode 200 which is located on the fabric-based substrate and is made of a fiber coated with a conductive composite ink containing a self-healing polymer and a metal-based fillers.

In addition, the fabric-based substrate and fiber-based nerve electrode may be self-bonded.

First, the bidirectional stretchable nerve interface according to an embodiment of the disclosure may include the fabric-based substrate 100.

The fabric-based substrate 100 may include a stretchable fabric 110 and a self-healing polymer film 120.

Here, the self-healing polymer film is coated to surround the stretchable fabric, or the stretchable fabric is located between respective layers of the plurality of self-healing polymer films.

The disclosure may maintain stretchability by locating the stretchable fabric between the self-healing polymer films.

Here, the stretchable fabric 110 may include one or more selected from the group consisting of polypropylene, nylon, polyvinylidene fluoride (PVDF), viscose, ultra-high-molecular-weight polyethylene (UHMWPE), polytetrafluoroethylene (PTFE), polyglycolic acid (PGA), catgut, catgut chromic, polyglactin 910, silk, poliglecaprone, polydioxanone, polyurethane, spandex, polyglycolic acid, and polyester.

For example, the stretchable fabric may be a silk-based fabric, and is not limited to the above-mentioned example, and any stretchable fiber with stretchability can be used without limitation.

In addition, the self-healing polymer may be an elastomer material whose backbone is any one selected from the group consisting of polydimethylsiloxane (PDMS), polyethyleneoxide (PEO), perfluoropolyether (PFPE), polybutylene (PB), poly(ethylene-co-1-butylene), poly(butadiene), hydrogenated poly(butadiene), poly(ethylene oxide)-poly(propylene oxide) block copolymer, random copolymer, and poly(hydroxyalkanoate).

The reason why the self-healing polymer includes an elastomer material whose backbone is any one selected from the group consisting of polydimethylsiloxane (PDMS), polyethyleneoxide (PEO), perfluoropolyether (PFPE), polybutylene (PB), poly(ethylene-co-1-butylene), poly(butadiene), hydrogenated poly(butadiene), and poly(ethyleneoxide)-poly(propylene oxide) block copolymer, random copolymer, and poly(hydroxyalkanoate) is because the polymer has self-healing property with stretchability.

Therefore, any polymer material with stretchability and self-healing property may be used as the self-healing polymer according to an embodiment of the disclosure without limitation.

Next, the bidirectional stretchable nerve interface according to an embodiment of the disclosure may include the fiber-based nerve electrode 200.

The fiber-based nerve electrode 200 is located on the fabric-based substrate 100, and a fiber 210 is coated with a conductive composite ink 220 containing a self-healing polymer and metal filler.

Here, the reason why the conductive fiber-based nerve electrode has a structure coated with the conductive composite ink is because, first, the fiber-based nerve electrode to be used as an electrode in the device is self-bonded with the substrate made by encapsulating both sides of a stretchable fabric with the self-healing polymer film.

Second, it is because the conductive ink made by dissolving the self-healing polymer has the advantage of being able to penetrate well not only the surface of the fiber but also the gaps between the bundles of fiber when coating the fiber strand, so the ink can be evenly coated across the entire fiber strand with a simple dipping process. The advantage of being able to penetrate the gaps between the fiber bundles also facilitates the fiber-to-fiber contact, making the electrode electrically and mechanically stable.

Third, it is because the conductive ink made by melting the self-healing polymer has the characteristics of being very stretchable, soft, and self-healing through various hydrogen bonds, so even if the coating process is applied to the fiber strands, the firmness of the fiber itself are not significantly affected.

Lastly, it is because, by coating the fiber strand with the conductive ink based on an inert metal that has properties suitable for the body, its stability as an implantable device that can be applied to the body is ensured, and due to the large charge delivery capacity of this material, it can provide stable electrical stimulation when used as a nerve interface.

In addition, by placing a durable fabric-based substrate and fiber-based nerve electrode within the stretchable self-healing polymer with soft tissue-like properties, it is possible to improve elasticity and tissue-like properties as well as mechanical and electrical durability.

Conventionally, much research has been conducted on the implantable devices using materials with mechanical properties similar to those of tissues, but these materials have limitations in maintaining electrical or mechanical properties when used for long periods of time. When these materials are used for a long time, there are problems with limited elasticity, deterioration of mechanical and electrical performance due to shape deformation and damage, but the structure in which the fiber is coated with conductive composite ink, according to the disclosure, can overcome these problems.

Specifically, the fiber may be a composite that includes one or more fibers selected from the group consisting of polypropylene, nylon (polyamide), PVDF, viscose, UHMWPE, PTFE, PGA, catgut, catgut chromic, polyglactin 910, silk, poliglecaprone, polydioxanone, polyurethane, spandex, polyglycolic acid and polyester.

For example, it may include silver-plated nylon fiber (conductive silver thread) and graphene-formed thread.

Here, the fiber may be used without limitation as long as it is a material that has durability, and elasticity, and is not limited to the materials described above.

In addition, the thickness of the fiber according to an embodiment of the disclosure may be 0.03 mm to 0.15 mm.

Here, the thickness of the fiber is not limited to the above-described examples, and the conductive fibers of various thicknesses may be used depending on the application of the bidirectional stretchable nerve fiber interface according to an embodiment of the disclosure.

In addition, the metal-based fillers may be a composite in which a metal nanoshell is coated on a metal flake, but it is not limited thereto and may be composed of a single metal or their alloys.

When the metal flake is coated with the metal nanoshell, the effect of preventing oxidation of the metal flake and outflow of metal flake ions can be achieved.

Here, the metal composite ink may be prepared by dissolving the polymer in an organic solvent capable of dissolving the polymer and mixing this solution with a conductive filler coated with a biocompatible and conductive nanoshell on the surface of the silver flake.

Here, the metal flake may include one or more metals selected from the group consisting of silver, copper, and aluminum.

Here, conductive nanoshell may play a role in preventing oxidation of silver ions in the stretchable nerve interface according to an embodiment of the disclosure.

Therefore, the material that may be used as the metal filler may be used without limitation as long as it is a metal that has conductive properties and may be well dispersed in an organic solvent-based solution made by dissolving the polymer.

In addition, the metal nanoshell may include one or more metals selected from the group consisting of platinum, gold, iridium, or their alloys.

Here, silver is oxidized in the living body and the silver ions of the silver thread and silver flake are released and are not biocompatible, so when the metal nanoshell is coated to provide biocompatibility, it will play a role in preventing oxidation of the silver.

Therefore, because the disclosure includes metal flakes and metal nanoshell, it can exhibit improved biocompatibility and charge delivery capacity.

In addition, the fabric-based substrate and fiber-based nerve electrode according to an embodiment of the disclosure may be self-bonded at room temperature without separate processing, and may be self-bonded without being limited to a specific temperature.

As a result, there is no need to add a separate adhesive material for bonding the fabric-based substrate and the fiber-based nerve electrode, so it can be manufactured simply, and can be self-healed at room temperature without a separate temperature change or external trigger such as light or additives, and can be easily applied to the nerve without a fixation tool such as suture due to the self-bonding property. Accordingly, it has effects that can be used in various environments of the living body.

In addition, the strain rate of the bidirectional stretchable nerve interface according to an embodiment of the disclosure may be 10% or more.

For example, the stretchable nerve interface according to one embodiment of the disclosure causes little change in electrical resistance even if the strain rate of 20% is repeatedly applied, even if the nerve is stretched or moved due to leg movement in case where the stretchable nerve interface is actually applied to the nerve, it can have high durability that can maintain stable electrical performance.

Therefore, the stretchable nerve interface according to an embodiment of the disclosure is biocompatible, has elasticity, has high electrical and mechanical durability, and has excellent electrical stimulation and nerve signal measurement performance.

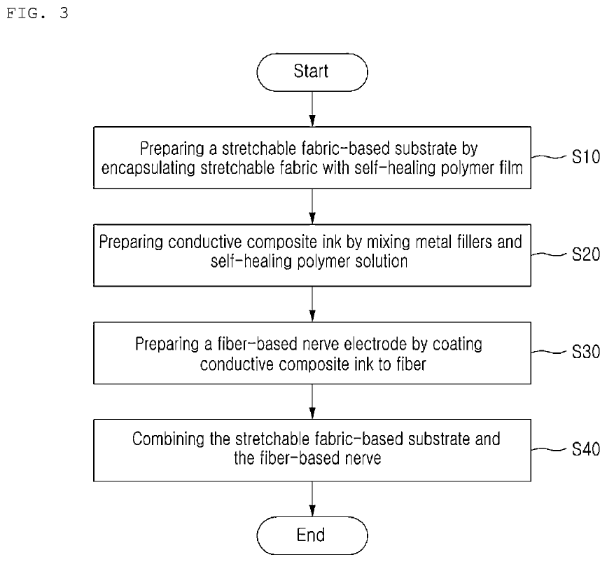

Referring to FIG. 3, a method for manufacturing a bidirectional stretchable nerve interface according to another embodiment of the disclosure will be described.

FIG. 3 is a flowchart showing a method for manufacturing a bidirectional stretchable nerve interface according to an embodiment of the disclosure.

Referring to FIG. 3, the method may comprise the steps of preparing a stretchable fabric-based substrate by encapsulating fabric with self-healing polymer films (S10);

preparing conductive composite ink by mixing a metal fillers and self-healing polymer solution (S20); preparing a fiber-based nerve electrode by coating the conductive composite ink to surround a fiber (S30); and preparing a stretchable nerve interface by combining the fabric-based substrate and the fiber-based nerve electrode (S40).

The first step may include preparing a stretchable fabric-based substrate by encapsulating fabric with self-healing polymer films(S10).

Here, the method of coating the self-healing polymer film to surround the stretchable fabric may include the steps of preparing a self-healing polymer film; placing the stretchable fabric between the self-healing polymer films and attaching the polymer films to the grid of the stretchable fabric by self-healing.

Here, the stretchable fabric 110 may include one or more selected from the group consisting of polypropylene, nylon, polyvinylidene fluoride (PVDF), viscose, ultra-high-molecular-weight polyethylene (UHMWPE), polytetrafluoroethylene (PTFE), polyglycolic acid (PGA), catgut, catgut chromic, polyglactin 910, silk, poliglecaprone, polydioxanone, polyurethane, spandex, polyglycolic acid, and polyester.

For example, the stretchable fabric may be a silk-based fabric, and is not limited to the above-mentioned example, and any stretchable fabric with elasticity can be used without limitation.

In addition, the self-healing polymer may be an elastomer material whose backbone is any one selected from the group consisting of polydimethylsiloxane (PDMS), polyethyleneoxide (PEO), perfluoropolyether (PFPE), polybutylene (PB), poly(ethylene-co-1-butylene), poly(butadiene), hydrogenated poly(butadiene), poly(ethylene oxide)-poly(propylene oxide) block copolymer, random copolymer, and poly(hydroxyalkanoate).

Here, any polymer material any polymer material with stretchability and self-healing property may be used as the self-healing polymer according to an embodiment of the disclosure without limitation.

The second step may include preparing conductive composite ink by mixing a metal fillers and self-healing polymer solution (S20).

In addition, the self-healing polymer may be one or more stretchable polymers selected from the group consisting of elastomer materials whose backbone is any one selected from the group consisting of polydimethylsiloxane (PDMS), polyethyleneoxide (PEO), perfluoropolyether (PFPE), polybutylene (PB), poly(ethylene-co-1-butylene), poly(butadiene), hydrogenated poly(butadiene), poly(ethylene oxide)-poly(propylene oxide) block copolymer, random copolymer, and poly(hydroxyalkanoate).

In addition, the metal-based fillers may be a composite in which a metal nanoshell is coated on a metal flake, but it is not limited thereto and may be composed of a single metal or their alloys.

The metal-based fillers in which the metal nanoshell is coated on the metal flake may be prepared by performing the steps of coating the surface of the metal flake with nanoshell of an inert metal; after dissolving the self-healing polymer in an organic solvent to prepare a polymer solution, dispersing the metal flake coated with the nanoshell in the polymer solution and thoroughly mixing the polymer solution.

In this case, the metal flake made of materials such as silver (Ag) has excellent conductivity, but may be easily oxidized, and thus silver ions may be released when inserted into the body, so the surface of the flake may be coated with the nanoshell of the inert metal.

Here, the metal flake may include one or more metals selected from the group consisting of silver, copper, and aluminum.

In addition, the metal nanoshell may include one or more metals selected from the group consisting of platinum, gold, iridium, or their alloys.

In addition, the thickness of the metal nanoshell may be 1 nm to 50 nm.

In addition, a solvent may be used to prepare the conductive composite ink according to an embodiment of the disclosure.

Here, the solvent used may be a material capable of dissolving the self-healing polymer, and specifically, chloroform, hexyl acetate (HA), or methyl isobutyl ketone (MIBK) may be used as the solvent, but the solvent is not limited to the above-mentioned examples.

Therefore, because the disclosure includes the metal flake and metal nanoshell, it can exhibit biocompatibility and charge delivery capacity.

The third step may include preparing a nerve electrode by dipping fiber into conductive composite ink (S30).

Here, the method of preparing the fiber-based nerve electrode by coating the conductive composite ink to surround the fiber may specifically include the steps of synthesizing the metal-based fillers; preparing conductive composite ink by mixing metal fillers and self-healing polymer solution; coating the conductive composite ink to surround the fiber by dipping a fiber in the conductive composite ink; and drying.

Here, the fiber may be a composite that includes one or more fibers selected from the group consisting of polypropylene, nylon, polyvinylidene fluoride (PVDF), viscose, ultra-high-molecular-weight polyethylene (UHMWPE), polytetrafluoroethylene (PTFE), polyglycolic acid (PGA), catgut, catgut chromic, polyglactin 910, silk, poliglecaprone, polydioxanone, polyurethane, spandex, polyglycolic acid, and polyester.

For example, it may include silver-plated nylon fiber (conductive silver thread) and graphene-formed thread.

Here, the fiber may be used without limitation as long as it is a material that has electrical conductivity and durability, and is not limited to the materials described above.

For example, conductive silver thread may be used as the conductive fiber.

The fourth step may include preparing the stretchable nerve interface by combining stretchable fabric-based substrate and the fiber-based nerve electrode. (S40)

In addition, the stretchable fabric-based substrate and the fiber-based nerve electrode according to an embodiment of the disclosure are characterized by self-bonding.

As a result, there is no need to add a separate adhesive material for bonding the stretchable fabric-based substrate and the fiber-based nerve electrode, so it can be manufactured simply, and can be self-healed at room temperature without a separate temperature change or external trigger such as light or additives. Accordingly, it has effects that can be used in various environments of the living body.

Therefore, it is possible to simply manufacture the nerve interface without complicated processes by using the self-bonding characteristics of the substrate and electrode.

In addition, the stretchable nerve interface may minimize tissue damage due to the biocompatibility and modulus matching with tissue of the conductive composite ink-coated fiber-based nerve electrode and stretchable fabric-based substrate, while being durable and stable even against extreme external deformation due to its center made of fiber and fabric. Also, it is made of the self-healing polymer, so it can be easily combined with a substrate when manufacturing the nerve interface.

Hereinafter, the disclosure will be described in more detail through manufacturing examples and experimental examples. These manufacturing examples and experimental examples are only for illustrating the disclosure, and the scope of the disclosure is not limited by these manufacturing examples and experimental examples.

### Manufacturing Example: Bidirectional Stretchable Nerve Interface Manufacturing Method

First, a silver flake-platinum nanoshell composite was prepared by coating the surface of the silver flake with a platinum nanoshell to cover and coat the silver flake (600 mg), using an aqueous solution prepared by dissolving 260 mg of chloroplatinic acid (H2PtCl6).

Next, thiol group was attached to the surface of the conductive silver flake coated with the platinum nanoshell so that the coated conductive silver flake could be well dispersed in a polymer-based organic solvent.

Next, 600 mg of the silver flake-platinum nanoshell composite was completely dissolved in 200 mg of the self-healing polymer and then mixed well to be evenly dispersed in 2 ml of chloroform (CHCl₃) to prepare the conductive composite ink.

Next, a 10 cm conductive fiber with a thickness of 0.03 to 0.04 mm was dipped in 2 ml of conductive ink for 5 seconds and dried for 5 second, and this dipping and drying process was repeated five times to coat the surface of the conductive thread with the conductive composite ink to prepare a fiber-based nerve electrode.

Next, a stretchable nerve interface was manufactured by installing the fiber-based nerve electrode in a desired shape on the fabric-based substrate.

### Experimental Example 1: Analysis of Electrical Properties of a Bidirectional Stretchable Nerve Interface According to Long-Term Deformation

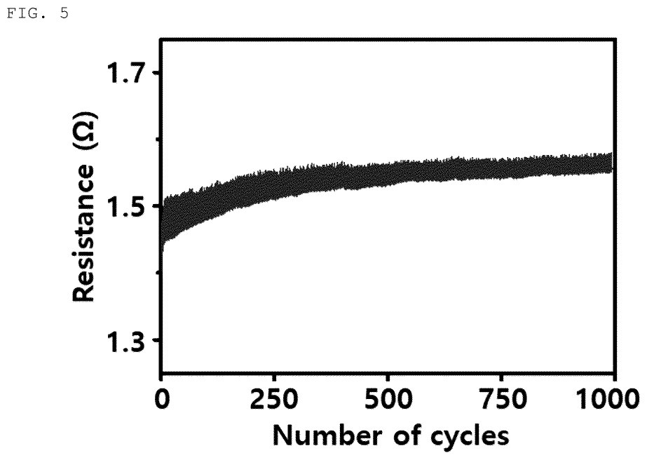

Referring to FIG. 5, the electrical characteristics of the nerve electrode according to long-term deformation will be described.

FIG. 5 is a graph showing a change in resistance of a bidirectional stretchable nerve interface according to an embodiment of the disclosure.

Referring to FIG. 5, in the case of a bidirectional stretchable nerve interface showing a strain rate of 20%, it was confirmed that it exhibited stable electrical characteristics without a large change in resistance despite a high strain rate, and maintained high conductivity stably (R/R₀ of 0.13 or less) even when repeatedly deformed more than 1000 times.

### Experimental Example 2: Electrical Stimulation Analysis of a Directional Stretchable Nerve Interface

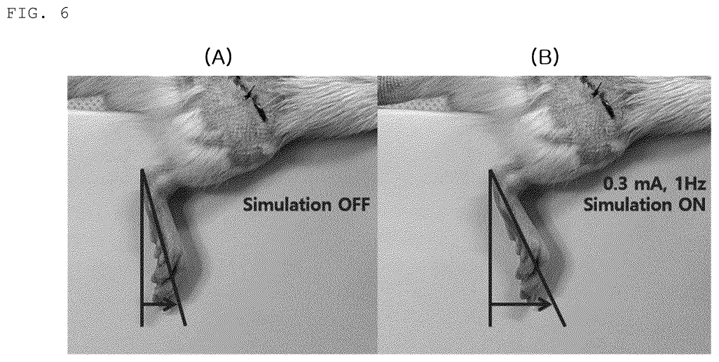

Referring to FIG. 6, the electrical stimulation performance of the bidirectional stretchable nerve interface will be described.

Referring to FIG. 6, when electrical stimulation of 0.3 mA, 1 Hz is applied to a rat, the leg angle of the rat increases, confirming that the bidirectional stretchable nerve interface according to an embodiment of the disclosure is capable of transmitting electrical signals.

### Experimental Example 3: Nerve Signal Analysis of a Bidirectional Stretchable Nerve Interface According to Electrical Stimulation Intensity

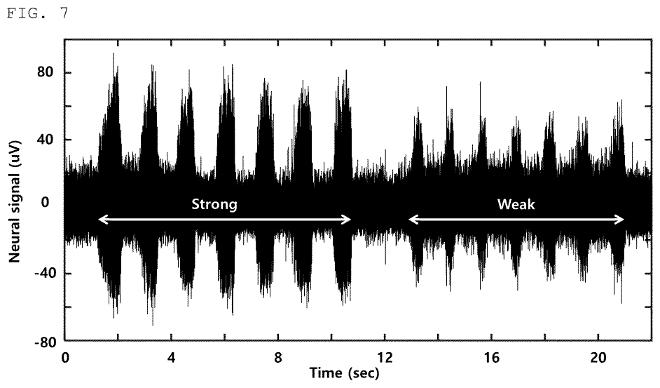

Referring to FIG. 7, nerve signals of the bidirectional stretchable nerve interface according to the electrical stimulation intensity will be described.

FIG. 7 shows data measuring changes in peripheral nerve signals according to sensation. Here, the nerve electrode was applied to the peripheral nerves near the thigh of a small rodent, and mechanical stimuli were applied to the paw.

Referring to FIG. 7, compound neural signals are measured by applying mechanical stimulation of different intensities to the sole of the foot in the same direction using a brush. When brushing the soles of the feet with a strong intensity, the signal amplitude may be large, and when brushing the soles with a relatively weak intensity, the signal amplitude may be small. FIG. 7 shows measurement data stimulated by performing the above process 7 times each.

Therefore, it can be confirmed from FIG. 7 that it is possible to measure neural signals according to various mechanical stimulation intensities.

The description of the disclosure is used for illustration and those skilled in the art will understand that the disclosure can be easily modified to other detailed forms without changing the technical spirit or an essential feature thereof. Therefore, the aforementioned exemplary embodiments are all illustrative in all aspects and are not limited. For example, each component described as a single type may be implemented to be distributed and similarly, components described to be distributed may also be implemented in a combined form.

The scope of the disclosure is to be defined by the scope of claims provided below, and all variations or modifications that can be derived from the meaning and scope of the claims as well as their equivalents are to be interpreted as being encompassed within the scope of the disclosure.

In the bidirectional stretchable nerve interface according to an embodiment of the disclosure, the substrate is composed of the stretchable fabric material and the biocompatible self-healing polymer so it has high durability and biocompatibility, the electrode is formed by coating highly conductive metal flake with metal nanoshell with high biocompatibility and charge delivery capacity, and then mixed with the self-healing polymer which has modulus matching with tissue to those of tissue and coated on conductive fibers. Therefore, the bidirectional stretchable nerve interface can provide a fiber-based bidirectional stretchable nerve interface that is flexible and has high electrical and mechanical durability while providing excellent stimulation and neural signal measurement performance.

In addition, the electrode according to an embodiment of the disclosure exhibits stable electrical characteristics with R/R₀ of 0.13 or less even in experiments in which the electrode is repeatedly deformed at a 20% stretching ratio over 1000 times. Therefore, it has the effect of stimulating nerves and measuring nerve signals while firmly maintaining electrical and mechanical properties without deforming its shape even during repetitive movements in the body.

The effects of the disclosure are not limited to the above-mentioned effects, and it should be understood that the effects of the disclosure include all effects that could be inferred from the configuration of the invention described in the detailed description of the invention or the appended claims.

### DESCRIPTION OF REFERENCE NUMERALS

100: stretchable fabric-based substrate
110: stretchable fabric
120: self-healing polymer
200: fiber-based nerve electrode
210: fiber
220: self-healable nanocomposite conductor

What is claimed is:

1. A bidirectional stretchable nerve interface comprising:
a stretchable fabric-based substrate in which a stretchable fabric is located between respective layers of a plurality of self-healing polymer films; and
a fiber-based nerve electrode which is located on the fabric-based substrate and is made of a fiber coated with a conductive composite ink containing a self-healing polymer and metal-based fillers.

2. The bidirectional stretchable nerve interface of claim 1, wherein the fabric-based substrate and the fiber-based nerve electrode are self-bonded.

3. The bidirectional stretchable nerve interface of claim 1, wherein the stretchable fabric includes one or more selected from the group consisting of polypropylene, nylon, polyvinylidene fluoride (PVDF), viscose, ultra-high-molecular-weight polyethylene (UHMWPE), polytetrafluoroethylene (PTFE), polyglycolic acid (PGA), catgut, catgut chromic, polyglactin 910, silk, poliglecaprone, polydioxanone, polyurethane, spandex, polyglycolic acid, and polyester.

4. The bidirectional stretchable nerve interface of claim 1, wherein the self-healing polymer is an elastomer material whose backbone is any one selected from the group consisting of polydimethylsiloxane (PDMS), polyethyleneoxide (PEO), perfluoropolyether (PFPE), polybutylene (PB), poly(ethylene-co-1-butylene), poly(butadiene), hydrogenated poly(butadiene), poly(ethylene oxide)-poly(propylene oxide) block copolymer, random copolymer, and poly(hydroxyalkanoate).

5. The bidirectional stretchable nerve interface of claim 1, wherein the fiber may be a composite that includes one or more fibers selected from the group consisting of polypropylene, nylon (polyamide), PVDF, viscose, UHMWPE, PTFE, PGA, catgut, catgut chromic, polyglactin 910, silk, poliglecaprone, polydioxanone, polyurethane, spandex, polyglycolic acid and polyester.

6. The bidirectional stretchable nerve interface of claim 1, wherein the metal-based fillers is a composite in which a metal nanoshell is coated on a metal flake.

7. The bidirectional stretchable nerve interface of claim 6, wherein the metal flake includes one or more metals selected from the group consisting of silver, copper, and aluminum.

8. The bidirectional stretchable nerve interface of claim 6, wherein the metal nanoshell includes one or more metals selected from the group consisting of platinum, gold, and iridium.

9. The bidirectional stretchable nerve interface of claim 6, wherein a thickness of the metal nanoshell is 1 nm to 50 nm.

10. The bidirectional stretchable nerve interface of claim 1, wherein a strain rate of the bidirectional stretchable nerve interface is 10% or more.

11. A method for manufacturing a bidirectional stretchable nerve interface, comprising the steps of:
preparing a stretchable fabric-based substrate by encapsulating stretchable fabric with self-healing polymer;
preparing conductive composite ink by mixing a metal-based fillers and self-healing polymer solution;
preparing a fiber-based nerve electrode by coating the conductive composite ink to surround a fiber; and
preparing a stretchable nerve interface by combining the stretchable fabric-based substrate and the fiber-based nerve electrode.

12. The method of claim 11, wherein in the step of preparing the stretchable fabric-based substrate, the stretchable fabric includes one or more selected from the group consisting of polypropylene, nylon, polyvinylidene fluoride (PVDF), viscose, ultra-high-molecular-weight polyethylene (UHMWPE), polytetrafluoroethylene (PTFE), polyglycolic acid (PGA), catgut, catgut chromic, polyglactin 910, silk, poliglecaprone, polydioxanone, polyurethane, spandex, polyglycolic acid, and polyester.

13. The method of claim 11, wherein in the step of preparing the stretchable fabric-based substrate, the self-healing polymer is an elastomer material whose backbone is any one selected from the group consisting of polydimethylsiloxane (PDMS), polyethyleneoxide (PEO), perfluoropolyether (PFPE), polybutylene (PB), poly(ethylene-co-1-butylene), poly(butadiene), hydrogenated poly(butadiene), poly(ethylene oxide)-poly(propylene oxide) block copolymer, random copolymer, and poly(hydroxyalkanoate).

14. The method of claim 11, wherein in the step of preparing the conductive composite ink, the metal-based fillers is a composite in which a metal nanoshell is coated on a metal flake.

15. The method of claim 14, wherein the metal flake includes one or more metals selected from the group consisting of silver, copper, and aluminum.

16. The method of claim 14, wherein the metal nanoshell includes one or more metals selected from the group consisting of platinum, gold, and iridium.

17. The method of claim 14, wherein a thickness of the metal nanoshell is 1 nm to 50 nm.

18. The method of claim 11, wherein in the step of preparing the fiber-based nerve electrode, the fiber is a composite that includes one or more fibers selected from the group consisting of polypropylene, nylon (polyamide), PVDF, viscose, UHMWPE, PTFE, PGA, catgut, catgut chromic, polyglactin 910, silk, poliglecaprone, polydioxanone, polyurethane, spandex, polyglycolic acid and polyester.

19. The method of claim 11, wherein in the step of preparing the stretchable nerve interface, the stretchable fabric-based substrate and the fiber-based nerve electrode are self-bonded.
